(19) United States
(12) Patent Application Publication
(10) Pub. No.: US 2002/0173704 A1
(43) Pub. Date: Nov. 21, 2002

(54) Apparatus and method for providing wireless data communication

(76) Inventors: Arthur E. Schulze, Wharton, TX (US); Waneta C. Tuttle, Albuquerque, NM (US)

Correspondence Address: Roberts Abokhair & Mardula, LLC, Suite 1000, 11800 Sunrise Valley Drive, Reston, VA, 20191-5302, US

(21) Appl. No.: 10/151,688

(22) Filed: May 20, 2002

Related U.S. Application Data
(60) Provisional application No. 60/292,065, filed on May 18, 2001.

(51) Int. Cl. A61B 5/00; H04M 11/00

(52) U.S. Cl. 600/300; 128/903; 128/904; 379/106.02

(57) ABSTRACT

A small sensor and instrument interface communication module provides the data acquisition circuitry required to gather various signals from sensors for a variety data, such as physiologic data from the human body or data from remote non-medical monitoring instrumentation, using analog, serial, and parallel digital inputs. The microprocessor and memory of the module are employed to condition those signals for interface to a variety of possible digital cellular phone or other wireless communication modules which are used to transmit these data into the digital cellular network for distribution on the Internet.

### RELATIONSHIP TO PRIOR APPLICATIONS

This application claims the benefit of U.S. Provisional Application No. 60/292,065, filed May 18, 2001 and hereby incorporated by reference.

### FIELD OF THE INVENTION

The present invention is drawn generally to data acquisition and communication devices. More particularly, the present invention is a device and method for acquiring, processing, and communicating a variety of data in a wireless mode over the Internet. It is useful in combination with co-pending U.S. patent applications 09/516,645 and 09/783,913, as well as for generalized acquisition and transfer of data to central Internet databases. The invention also allows control of the data acquisition parameters over the Internet.

### BACKGROUND INFORMATION

Presently, most data, including data from medical monitoring equipment and non-medical monitoring instrumentation, is transmitted to central locations from remote locations using telephone lines. In prior art systems, such as U.S. Pat. Nos. 5,307,263 and 5,997,476, a modem is used to modulate and demodulate digital data for transmission over the analog phone lines. The process typically involves a fairly time-consuming “handshaking” process to coordinate the transmission.

A purpose of the present device is to enable bi-directional communication between sensors and voice data acquisition devices connected to any patient or instrument (whether in motion or stationary) and a host server on the Internet in a wireless manner with generalized geographic coverage without use of a modem. The Sensor and Instrument Interface Communication Module (SIICM) permits monitoring of the data anywhere in the coverage map of a digital cellular telephone network, satellite-based networks, or other wireless networks by providing the bi-directional interface between sensors located on a patient, instrument, or equipment and a cellular telephone system or other wireless network. Data can be collected and archived from this interface and the wireless device can be accessed remotely through Internet-connected devices to configure the data acquisition device and to acquire such data. Digital on-board storage of data as well as direct two-way voice communications are also parts of the system; these parts being redundant to the Internet interface. Two-way command messaging and software modification are also provided.

### BRIEF SUMMARY OF THE INVENTION

It is an object of the invention to allow access to real-time or stored data for display and interpretation by medical care providers and other data analysts with Internet access.

It is a further object of the invention to provide a new form of connectivity/biotelemetry that allows physiologic and other data transfer from sensors and instruments located anywhere that digital cellular technology or digital satellite connectivity has been deployed.

It is another object of the invention to enable direct data archiving of large amounts of data directly over the Internet using new techniques (such as XML) with privacy and security issues addressed through Virtual Private Network (VPN) technology or other encryption procedures.It is another object of the invention to provide for automatic data collection without direct interaction by the user, remote instrument or patient.

It is another object of the invention to provide a device that contains circuitry for self-test of various subsystems and sensors of a medical or other monitoring device and circuitry for communication of troubleshooting information directly to a central Internet database in the event that a system problem occurs during use.

It is another object of the invention to provide a SIICM that contains alarms for monitored data that can be pre-set by external means, including over the Internet.

It is another object of the invention to provide communication means that can be optimized to fit common cellular calling and rate plans so as to minimize cost and airtime usage.

It is yet another object of the invention to provide the capabilities of the present invention while leveraging existing data acquisition equipment, sensors, digital communication equipment and networks, as well as incorporate the present SIICM into new instrumentation.

One of the major objects of the invention is to provide a smart sensor interface wherein sensor, instrument, or equipment characteristics are stored in the memory of the SIICM or are stored in the Central Internet Database which configures the SIICM device interface by remote control.

### BRIEF DESCRIPTION OF THE DRAWINGS

FIG. 1 illustrates a schematic diagram of the present invention.

### DETAILED DESCRIPTION OF THE INVENTION

A small sensor interface device (Sensor Instrument Interface Communication Module or SIICM) provides the data acquisition circuitry required to gather various signals from sensors for a variety data (including physiologic data from the human body or data from remote non-medical monitoring instrumentation) and to condition those signals for interface to a variety of possible digital cellular phone or other wireless communication modules which are used to transmit these data into the digital cellular network for distribution on the Internet.

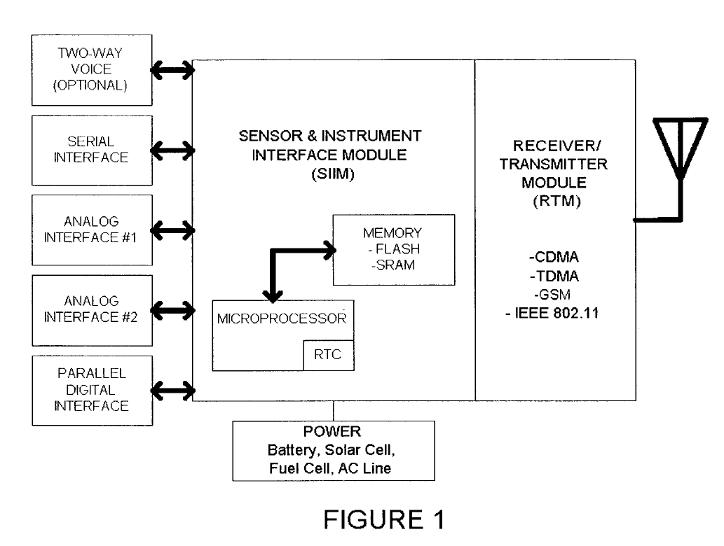

FIG. 1 is a sketch of the essential features of the Wireless Data Communicator, comprised of a Sensor Instrument Interface Communication Module (SIICM) connected to a Receiver/Transmitter Module (RTM) which provides the wireless interface to the Internet. The sensors can be any of a wide variety of physical and biosensors generally used to detect signals or variables from the (1) human body, (2) instruments, (3) equipment, (4) environment, etc. Sensors and instruments used in measuring clinically relevant data are of particular interest for use in this system. Such data include electrocardiogram, temperature, respiration, acceleration, audio, oximetry, blood glucose, body weight, capnogram, geographic position (GPS), blood pressure, keyboard, pipeline pressure, etc. The RTM can contain a variety of wireless communication protocols; e.g., CDMA, TDMA, GSM, Bluetooth, IEEE 802.11a, b, g, etc.

Power can be supplied from a variety of sources that include batteries, solar cells, fuel cells, AC lines, etc. Specialized sensor interface modules can be plugged into the SIICM to interface with a wider variety of sensors, instruments, or equipment.

The wireless data communicator (comprised of a SIICM and RTM and other interface modules, as required) can be distributed for attachment to a wide variety of sensors, instruments, and equipment or for incorporation in the design of such devices. In this manner, any device which acquires data can be converted into a wireless data communication device (by the use of the Wireless Data Communicator) that will transfer these data directly to the Internet. The exact configuration of these modules can be in a variety of packages, including the familiar PC card (PCMCIA form).

The SIICM is designed to recognize the characteristics of the sensor, instrument, or equipment to which it is attached and to encode the transmitted data in a manner that will allow the Central Internet Database to interpret and display the data. Since the Wireless Data Communicator has two-way digital communications capability, a smart sensor interface (similar to “plug and play”) can be implemented either in the memory of the SIICM or the SIICM can be configured remotely by commands from the Central Internet Database to recognize the interface.

Unique Features

Allows access to real-time or stored data for display and interpretation by medical care providers and other data analysts with Internet access

A new form of connectivity/biotelemetry which allows physiologic and other data transfer from sensors and instruments located anywhere that digital cellular technology or digital satellite connectivity has been deployed

Enables direct data archiving of large amounts of data directly over the Internet using new techniques (such as XML) with privacy and security issues addressed through Virtual Private Network (VPN) technology or other encryption techniques.

Designed for automatic data collection without direct interaction by the user or patient

Contains circuitry for self-test of the various subsystems and sensors and communication of troubleshooting information directly to patient the Central Internet Database in the event that a system problem occurs during use

Contains alarms on the monitored data that can be pre-set by external means (and over Internet, if desirable)

Communication rates can be optimized to fit common cellular calling and rate plans to minimize cost and airtime usage

Recognizes the characteristics of any of a series of pre-defined sensors, instruments, or equipment to which it is attached and provides an automated interface of these characteristics to the data acquisition and communications system

Wireless Data Communicator interfaces with existing data acquisition equipment and sensors

Cellular telephone equipment is already deployed which will enable the use of this system

The SIICM and RTM can be attached to existing instrumentation or built-in to new instruments

Interfaces

Analog Instruments can include those to measure:

ECG

Temperature

Respiration

Acceleration

Sound (microphone)

Flow

Pressure

Serial Instruments can include those to measure:

Pulse oximetry

Glucose

Body weight

Capnogrphy

Spirometry

Digital inputs can include:

Keyboard

GPS

Blood pressure

Although disclosed with respect to particular embodiments, one of ordinary skill in the art will understand that numerous modifications can be made wothout departing from the scope of the invention, which is limited solely by the claims, below.

We claim:

1. A sensor instrument interface communication module comprising:
a microprocessor;
memory means connected to said microprocessor;
circuitry to interface with receiver/transmitter module;
at least one analog input means;
at least one serial input means; and
at least one parallel digital input means.
